(19) United States
(12) Patent Application Publication
(10) Pub. No.: US 2023/0338629 A1
(43) Pub. Date: Oct. 26, 2023

(54) DECOMPRESSION SYSTEM FOR USE IN TREATING PNEUMOTHORAX

(71) Applicants: Jonathan M.E. Aho, Rochester, MN (US); Michael Gellatly, Miami Springs, FL (US)

(72) Inventors: Jonathan M.E. Aho, Rochester, MN (US); Michael Gellatly, Miami Springs, FL (US)

(21) Appl. No.: 18/134,899

(22) Filed: Apr. 14, 2023

Related U.S. Application Data
(60) Provisional application No. 63/334,079, filed on Apr. 22, 2022.

(51) Int. Cl. A61M 1/04

(52) U.S. Cl. CPC A61M 1/04; A61M 2205/0227; A61M 2205/3341

(57) ABSTRACT

The present invention relates to systems and components for the rapid decompression of pneumothorax. The system includes a needle and novel indicator assembly. The indicator assembly includes valve assembly and color changing member that responds to CO2, visually indicating that the system has been placed in an appropriate location and that decompression is occurring.

### BACKGROUND OF THE INVENTION

Thoracostomy procedures have been performed for many years to deliver therapeutics and/or remove fluids from the pleural cavity (or space) housing the lungs. One such reason to perform these procedures involves the buildup of fluids (in particular gasses such as air) in the pleural space, which can impair respiration by restricting the normal inflation of the lungs in a condition known as pneumothorax. When a pneumothorax occurs and is allowed to persist and continues to build pressure without resolution, it alters heart function, leading to a catastrophic cardiopulmonary arrest. Resolution of the pneumothorax typically involves inserting and positioning a chest tube or catheter through the chest wall and into pleural cavity. These procedures are typically performed in a hospital setting and may utilize thorascocopic or ultrasound visualization for proper placement of the catheter which can be difficult without substantial experience. Since the development of pneumothoraces can occur as a result of trauma or other conditions (outside of a hospital setting), there is a need for simple, easy to use, low cost system (or kit) that can be utilized by emergency personnel or even by inexperienced non-medical responders (not in a hospital or clinic setting), or personnel in a hospital or clinic setting under austere circumstances to provide immediate effective treatment while confirming accurate system positioning and that rapid decompression is occurring.

A prototype needle decompression system for treatment of pneumothorax has been described in published article, “Needle Decompression of Tension Pneumothorax with Colorimetric Capnography” CHEST 2017; 152(5):1015-1020 by the inventors, Aho et. al. While the prototypes provided encouraging initial results, additional improvements and modifications are needed for the system.

### SUMMARY

In accordance with one aspect of the invention there is provided a medical device system for rapidly treating pneumothorax. More particularly, there is provided a pneumothorax decompression system which includes an elongate tubular needle having proximal and distal ends with a lumen extending there between, a connector positioned at the needle's proximal end and a removably attachable indicator assembly. For convention and general context, distal and proximal are relative positions with distal being closer to the patient and proximal being farther away from the patient. The needle preferably has a sharpened distal end for piercing the chest wall of a patient. The indicator assembly includes a tubular housing having distal and proximal ends with a lumen extending there between, a valve assembly and a color changing portion. The indicator assembly includes a connector positioned at the distal end of the tubular housing for releasably connecting the assembly to the proximal end of the needle. The indicator assembly may also include a connector positioned at the proximal end of the tubular housing to facilitate releasably connecting the indicator assembly to auxiliary equipment. The valve assembly is preferably positioned adjacent the distal end of the indicator assembly and includes a valve that has an open configuration and a closed configuration to allow or restrict/prevent fluid flow between the distal and proximal ends of the tubular housing.

In accordance with another aspect of the invention there is provided a valve assembly that includes a valve that is normally biased in a closed configuration, preventing or restricting flow between the ends of the tubular housing. The valve of the valve assembly, also has a valve opening pressure that allows the valve to move from its normally biased closed configuration to an open configuration when the pressure on the distal side of the valve assembly exceeds the valve opening pressure. When this valve opening pressure is exceeded, the valve moves from its biased closed configuration to its open configuration allowing fluid flow from the distal end of the tubular housing, through the housing to the proximal end of the housing and out then to the ambient environment.

In accordance with yet another aspect of the decompression system there is provided a valve assembly that includes a valve having a open configuration when fluid pressure is applied to the distal side of the valve (or negative is pressure applied to the proximal side of the valve) and that applied pressure exceeds a first valve threshold pressure. The valve assembly has a closed configuration when fluid pressure is applied to the proximal side of the valve (or negative pressure is applied to the distal side of the valve) and the applied pressure exceeds a second valve threshold pressure. The first and second valve threshold pressure may be equal in magnitude or differ, with one threshold pressure being greater than the other due to the valve material, configuration and or assembly configuration.

In accordance with another embodiment of the decompression system there is provided a valve assembly that includes a one-way valve. The one-way valve only allows fluid to move in the direction from the distal end indicator assembly to the proximal end of the assembly. Upon applied fluid pressure from the proximal end of the indicator assembly, the valve assembly moves to a closed configuration, preventing fluid flow distal to the valve assembly.

The color changing portion of the indicator assembly is positioned within the lumen of the tubular housing between the valve assembly and the proximal end of the housing. The color changing portion preferably takes the form of a material that changes color in the presence of CO2. Readily available suitable materials may include capnography paper. Other pH sensitive or CO2 gas specific materials may also be suitable for use in the color changing portion. Typically, these materials change color over a period of time when exposed to CO2. The rate of change of the color of these materials is typically associated with the concentration of the CO2 in contact with the material and the material surface area. As a part of an indicator assembly for use in a rapid decompression system for pneumothorax, it is important to obtain a color change (indicating CO2 presence) as quickly as possible, to insure proper system placement and that decompression is occurring. Additionally, it is preferable that the color changing portion be reversible. This means that the color change of the color changing portion that occurs at a threshold concentration of CO2 (denoting CO2 presence), returns to its previous color should the concentration of CO2 diminish below the threshold concentration. This aspect is important to insure that the CO2 is flowing during the entire decompression process. For instance, should a blockage occur in the conduit leading to the indicator assembly, a reversing color change of the color changing portion would provide an immediate signal or notification to operator that some action must be taken to properly continue the decompression process.

In accordance with yet another embodiment of a decompression system there is provided an indicator assembly having a color changing portion or an indicator changing portion that changes its visual appearance from a first state to a second state upon the detection of CO₂. The indicator changing portion may incorporate sensor detection methods, modalities, technologies and components that include spectroscopy, lasers, and sensors that detect changes in gas electrical resistivity or conductivity and other suitable detectors. Upon detection of the presence of CO₂, the indicator changing portion changes its visual appearance from an initial first state to a second state alerting the user that CO₂ is present and that the system is properly positioned and functioning.

In accordance with one aspect of the decompression system, there is provided an indicator assembly which is formed from a transparent material. Suitable materials include glass and more preferably transparent biocompatible plastics such as acrylics, polystyrenes, polycarbonates, in addition to blends and copolymers. Additional suitable materials include translucent materials that allow for visibility of the color changing portion. These translucent materials may include crystalline and semi-crystalline polymers such as a wide array of polyolefins that have a very small crystal size or are processed in such a way that they provide visibility required.

In accordance with another aspect of the decompression system there is provided an indicator assembly that utilizes a valve that has a very low valve opening pressure.

In accordance with still another aspect of the decompression system there is provided an indicator assembly that maximizes the surface area of the color changing portion in potential contact with CO₂ The tubular housing of the indicator assembly may include internal ridges, bumps, dimples, grooves or surface roughness to support a color changing portion formed from a color changing material (rolled thin sheet) such that fluid flow in the color changing portion contacts first and second sides of the color changing material as opposed to only one side of the color changing material. Alternatively, the tubular housing may have a smooth internal surface and the color changing material may be formed to have bump, dimples, grooves, ridges or the like, which provides a fluid flow path between the side of the color changing material directly adjacent or in contact with the housing.

### BRIEF DESCRIPTION OF THE DRAWINGS

FIG. 1 is a perspective view of a rapid decompression system for pneumothorax including a needle and an attached indicator assembly.

FIG. 2A is a side view of an indicator assembly.

FIG. 2B is an enlarged cross-sectional view of the indicator assembly shown in FIG. 2A

FIG. 3A is a side view of another embodiment of an indicator assembly.

FIG. 3B. is an enlarged cross-sectional view of the indicator assembly depicted in FIG. 3A.

FIG. 4A is a side view still of another embodiment of an indicator assembly.

FIG. 4B is an enlarged cross-sectional view of the indicator assembly shown in FIG. 4A.

FIG. 4C is an enlarged perspective cross-sectional view of the indicator assembly shown in FIG. 4A.

### DETAILED DESCRIPTION

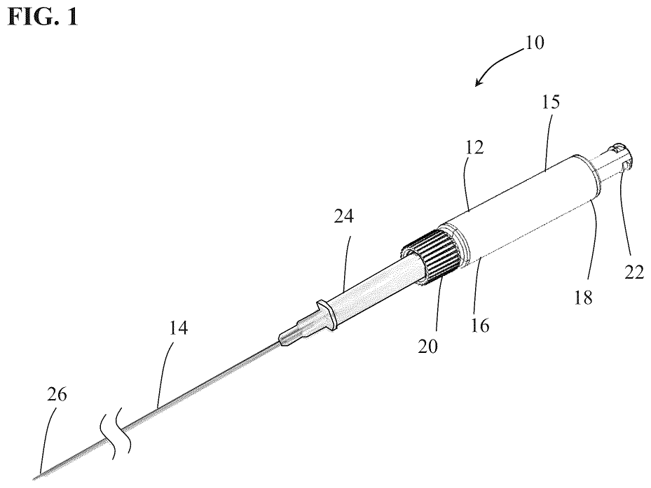

FIG. 1 illustrates an easy to use, low cost pneumothorax rapid decompression system 10 that includes an indicator assembly 12 coupled to needle 14. The indicator assembly 12 takes the form of a tubular housing 15 having a distal end 16 and a proximal end 18 and a lumen extending therebetween. Distal connector 20 and proximal connector 22 are secured at the housing ends, respectively. Positioned within the tubular housing 15 are a valve assembly and a color changing component that changes color in the presence of CO₂. Needle 14 has a proximal connector 24, a distal end 26 and a lumen extending therethrough. Needle 14 is coupled to indicator assembly 12 such that the proximal connector 24 is coupled to distal connector 20 in fluid tight engagement whereby the lumen of the needle is contiguous with the lumen of the tubular housing 15. The needle may be of a standard type for use thoracostomy procedures, a more specialized type incorporating the functionality of a Veress needle, or any suitable medical cannula or catheter. When the needle is inserted through the chest tissue in patient with a pneumothorax and is in the proper location the pressurized air flows through the needle, into the indicator assembly causing the valve assembly to open. This allows the flowing air (containing CO₂ levels above ambient air) to contact the color changing component causing the color to change as it flows out the proximal end of the indicator assembly. Alternatively, negative pressure may be applied to the proximal end of the indicator assembly (via a removably connected syringe or suitable alternative) to draw fluid through the needle and through the color changing portion. The valve assembly is preferably a one way valve to restrict airflow between the ends of the indicator assembly and thus preventing ambient air from potentially entering the body should the needle be improperly positioned or upon respiration.

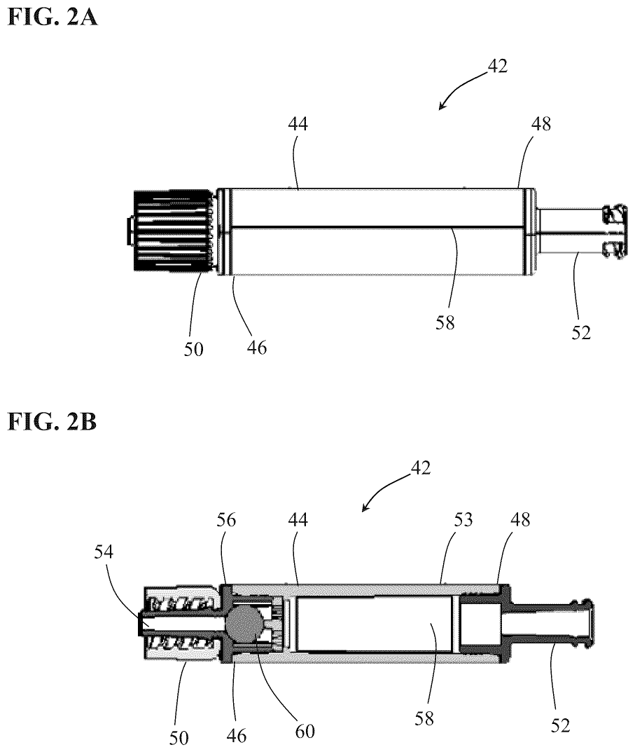

FIGS. 2A and 2B illustrate in more detail, a novel indicator assembly that may be utilized in a pneumothorax decompression system. The indicator assembly 42 has a tubular housing 44 with a distal end 46 and proximal end 48 and a lumen extending therebetween. The distal and proximal ends include connectors 50 and 52 respectively. Connector 50 is adapted to couple to a needle or other cannula that may be inserted into the chest of a patient, while connector 52 is adapted to couple to auxiliary equipment. The connectors 50 and 52 are typically molded components that are fixedly secured to the housing 44 during manufacture of the indicator assembly 42 using glue, adhesives, welding or other joining techniques known in the art. As depicted in FIG. 2B, indicator assembly 42 includes a valve assembly 56 positioned within the lumen 54 of tubular housing 44 adjacent distal connector 50. Within the housing 44, between the valve assembly 56 and proximal end 48, there is a color changing portion 58. The color changing portion 58 may typically take the form of a rolled sheet of capnography paper although other materials and configurations may be suitable. The color changing portion 58 is generally positioned adjacent to the wall 53 of the tubular housing 44. As previously discussed, the housing 44 is preferably formed from transparent material so that the change in color of the color changing portion 58 is visible through the housing wall 53. The valve assembly 56 for the indicator assembly 42 preferably has a low valve opening pressure and to accomplish this, ball valve 60 is utilized.

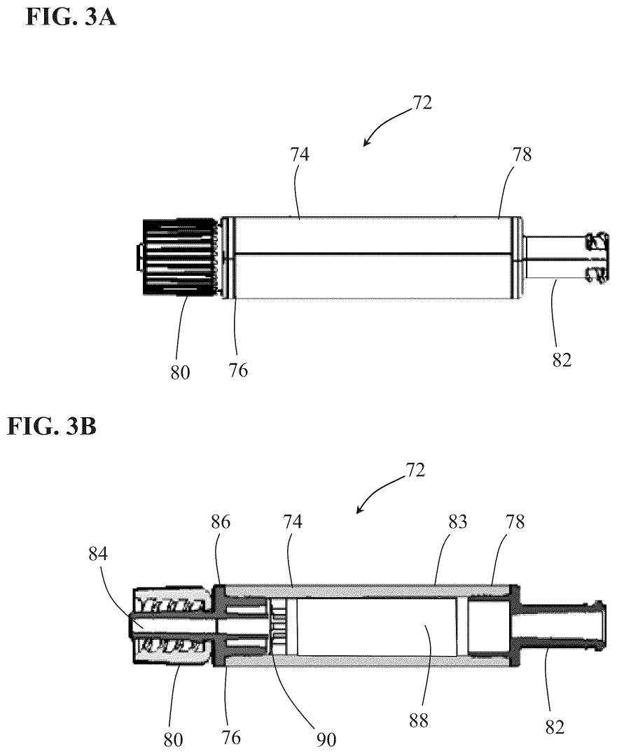

Similar to the previously described indicator assembly, FIGS. 3A and 3B, show an indicator assembly in which a different type of valve is utilized. The indicator assembly 72 has a tubular housing 74 with a distal end 76 and proximal end 78 and a lumen extending therebetween. The distal and proximal ends include connectors 80 and 82 respectively. Connector 80 is adapted to couple to a needle or other cannula that may be inserted into the chest of a patient, while connector 82 is adapted to couple to auxiliary equipment. As depicted in FIG. 3B, indicator assembly 72 includes a valve assembly 86 positioned within the lumen 84 of tubular housing 74 adjacent distal connector 80. Within the housing 74, between the valve assembly 86 and proximal end 78, there is a color changing portion 88. The color changing portion 88 may typically take the form of a rolled sheet of capnography paper although other materials and configurations may be suitable. The color changing portion 88 is generally positioned adjacent to the wall 83 of the tubular housing 74. As previously discussed, the housing is preferably formed from transparent material so that the change in color of the color changing portion 88 is visible through the housing wall 83. The valve assembly 86 for the indicator assembly 72 preferably has a low valve opening pressure and to accomplish this, a membrane valve 90 is utilized. Valves like membrane valve 90 may be more compact relative to ball valves, thereby improving manufacturability. Membrane valve 90 also has benefits over other valve types which include the ability to tune the threshold pressure for opening and closing the valve assembly, flexibility in choice of materials and sizes which can improve functionality, performance and manufacturability. Additionally, the valve (or a separate valve assembly member) may be formed of, or incorporate absorbent materials that can capture any bodily liquids that may enter the needle (from the chest cavity) to prevent the liquids from fouling the color changing portion of the indicator assembly.

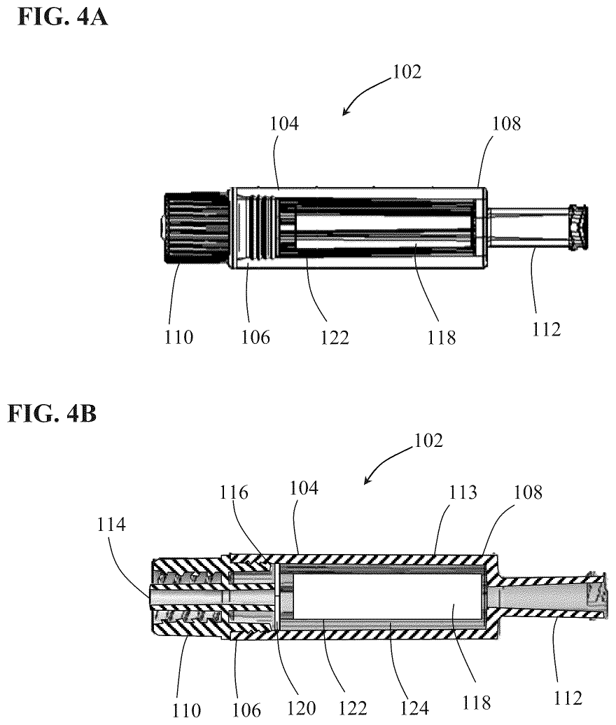

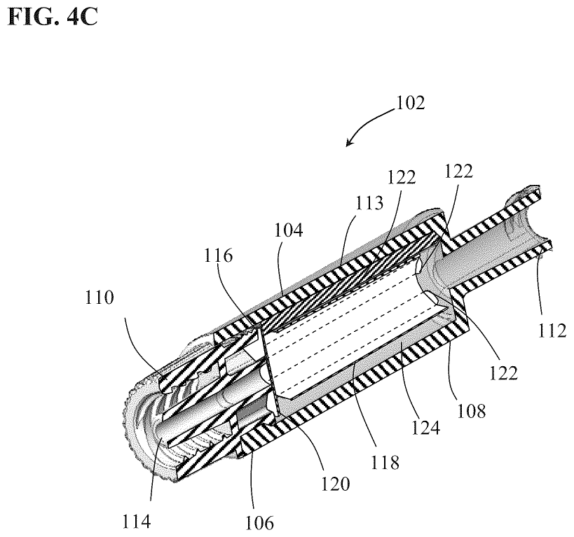

Another embodiment of a novel indicator assembly is shown in FIGS. 4A though 4C. The indicator assembly 102 has a tubular housing 104 with a distal end 106 and proximal end 108 and a lumen extending therebetween. The distal and proximal ends include connectors 110 and 112 respectively. Connector 110 is adapted to couple to a needle or other cannula that may be inserted into the chest of a patient, while connector 112 is adapted to couple to auxiliary equipment. While connector 110 is a molded component that is securely fixed to the tubular housing 104 during the manufacturing process of the indicator assembly 102, connector 112 is integrally formed with tubular housing 104, thereby eliminating a component and assembly step to improve manufacturability. As illustrated in FIGS. 4B and 4C, indicator assembly 102 includes a valve assembly 116 positioned within the lumen 114 of tubular housing 104 adjacent distal connector 110. Within the housing 104, between the valve assembly 116 and proximal end 108, there is a color changing portion 118. The color changing portion 118 may typically take the form of a rolled sheet of capnography paper although other materials and configurations may be suitable. Unlike the prior discussed assemblies, tubular housing 104 has a wall 113 that includes a plurality of internal ridges 122. The color changing portion 118 is generally positioned within the tubular housing 104 contacting ridges 122 and forming adjacent channels 124. As previously discussed, the housing is preferably formed from transparent material so that the change in color of the color changing portion 118 is visible through the housing wall 113. The valve assembly 116 for the indicator assembly 102 preferably has a low valve opening pressure and to accomplish this, a membrane valve 120 is utilized. Like the aforementioned indicator assemblies, the valve (or a separate valve assembly member) may be formed of, or incorporate absorbent materials that can capture any bodily liquids that may enter the needle (from the chest cavity) to prevent the liquids from fouling the color changing portion of the indicator assembly.

While the addition of the ridges 122 and adjacent channels 124 within the lumen 114 of tubular housing 104 appear to be an aesthetic change, this construction offers significant functional benefits. Prior discussions about how the color changes in the color changing portion indicate that the color change is related to the concentration of CO₂ present and the duration it is in contact. In previously discussed embodiments of the indicator assembly, the color changing portion took the form of a rolled sheet that had the inner surface of the sheet in contact with the air flow coming from the body. The CO₂ must saturate the paper before the change is visible on the side of the paper adjacent housing wall. The ridge and channel construction of indicator assembly 102 offers fluid flow onto both sides of the paper maximizing the contact surface area, resulting in a faster color change. While the ridges 122 are shown extending longitudinally they may be of any suitable configuration including spirals or angles. Conversely, instead of raised ridges, the tubular housing wall may incorporate grooves in the wall in various geometric configurations to provide a similar benefit.

Novel devices, systems and methods have been disclosed to perform decompression procedures. While multiple embodiments of indicator assemblies have been discussed having different elements, it should be understood that various modifications including the substitution of elements or components which perform substantially the same function in the same way to achieve substantially the same result may be made by those skilled in the art without departing from the scope of the claims which follow.

1. A pneumothorax decompression system comprising:
a tubular needle having first and second ends and a lumen extending there between and a connector affixed to one of said ends and,
an indicator assembly including a tubular housing member having first and second ends and a lumen extending there between, a valve assembly secured within said housing member adjacent said first end and a color changing member having a first visual state and a second visual state being positioned within the lumen of said housing member between said valve assembly and said second end,
said needle being coupled to said housing member first end such that the lumen of said needle and said valve assembly are in fluid tight engagement and said color changing member is visually responsive to CO₂, such that when said color changing member is in said first state and CO2 containing fluid flows through said needle lumen contacting said color changing member, said color changing member moves from said first state to said second state providing a visible visual appearance different from said first state.

2. A pneumothorax decompression system according to claim 1 wherein said valve assembly includes a valve having a low valve opening pressure.

3. A pneumothorax decompression system according to claim 1 wherein said valve assembly includes a membrane valve.

4. A pneumothorax decompression system according to claim 1 wherein said valve assembly is fixedly secured to said tubular housing member.

5. A pneumothorax decompression system according to claim 1 wherein said first and second ends of said housing member include a connector.

6. A pneumothorax decompression system according to claim 1 wherein said housing member has a wall that includes ridges that define adjacent channels.

7. A pneumothorax decompression system according to claim 6 wherein said ridges and channels extend longitudinally within the lumen of said housing member.

8. A pneumothorax decompression system according to claim 6 wherein said color changing member is supported by said ridges.

9. A pneumothorax decompression system according to claim 1 wherein said color changing member takes the form of a cylinder.

10. A pneumothorax decompression system according to claim 1 wherein said housing member is formed of transparent material.

11. A pneumothorax decompression system according to claim 1 wherein said indicator assembly includes liquid absorbing materials.

12. A pneumothorax decompression system according to claim 1 wherein said color changing member is reversible such that when in said second state and CO2 is removed said member moves from said second state to said first state.

13. An indicator assembly for use in pneumothorax decompression procedures comprising:
a tubular housing having first and second ends and a lumen extending there between,
a valve assembly including a valve having a low valve opening pressure, being positioned within and fixedly secured to said tubular housing first end, and
a color changing member having a first color and a second color, wherein said color changing member moves from said first color to said second color when exposed to CO2, positioned within the lumen of said housing between said valve assembly and said housing second end, said valve assembly controlling the flow of fluid through said second end to said first end.

14. An indicator assembly system according to claim 13 wherein said housing has a wall that includes ridges that define adjacent channels.

15. An indicator assembly system according to claim 14 wherein said ridges and channels extend longitudinally within the lumen of said housing.

16. An indicator assembly system according to claim 14 wherein said color changing member is supported by said ridges.

17. An indicator assembly system according to claim 14 wherein said valve assembly includes a membrane valve.

18. An indicator assembly for use in pneumothorax decompression procedures comprising:
a tubular housing having a wall, first and second ends and a lumen extending there between, said housing wall having a plurality of raised and non-raised portions positioned within said lumen, and,
a color sensitive member positioned within the lumen of said housing between said said housing first and second ends, said color sensitive member contacting at least some of said raised portions and forming a fluid flow path between said color sensitive member and said non-raised portions, said color sensitive member having a first color, a second color and being moveable between said first and second colors, such that when said sensitive member is exposed to CO2, said color sensitive member moves from said first color to said second color.

19. An indicator assembly system according to claim 18 wherein said first and second ends include connectors.

20. An indicator assembly system according to claim 18 wherein said color sensitive member operates reversibly such that said color sensitive member moves from said second color to said first color when CO2 exposure is reduced.
